(19) United States
(12) Patent Application Publication
(10) Pub. No.: US 2004/0170675 A1
(43) Pub. Date: Sep. 2, 2004

(54) Noninvasive method for treating cellulite through transdermal delivery of calcium channel blocker agents and medicament for use in such method

(76) Inventor: W. Jerry Easterling, San Antonio, TX (US)

Correspondence Address: David G Henry, 900 Washington Avenue, PO Box 1470, Waco, TX, 76703-1470, US

(21) Appl. No.: 10/475,434

(22) PCT Filed: Apr. 19, 2002
(86) PCT No.: PCT/US02/12433

Related U.S. Application Data
(63) Continuation-in-part of application No. 09/839,605, filed on Apr. 20, 2001, now abandoned.
Application No. 09/839,605 is a continuation-in-part of application No. 09/514,796, filed on Feb. 28, 2000, now abandoned.
Application No. 09/514,796 is a continuation-in-part of application No. 09/128,103, filed on Aug. 3, 1998, now Pat. No. 6,031,005.

(51) Int. Cl. A61K 9/70

(52) U.S. Cl. 424/449

(57) ABSTRACT

The invention is of a novel, noninvasive method for treating cellulite involving the topical application of one or more calcium channel blocker agents suspended in one or more carriers with penetration enhancing agent's to effect transdermal delivery of the calcium channel blocker agent(s). Periodic, topical application of the medicaments taught here in will effect, usually within a few months, a substantial reduction or elimination of palpable cellulite.

This is a continuation-in-part application with respect to U.S. application Ser. No. 09/839,605 filed 20 Apr. 2001 (20.04.01) from which application under the Patent Cooperation Treaty.

### BACKGROUND OF THE INVENTION

1. Field of the Invention

The present invention relates to the elimination or mitigation of lipedema or “cellulite”.

2. Background Information

Cellulite is a colloquial term for deposits of fat and fibrous tissue causing dimpling of the overlying skin 2. SYN lipoedema. (Stedman's Medical Dictionary, 26th Edition, p. 307). Lipodystrophy refers to a defective metabolism of fat. (Stedman's Medical Dictionary, 26th Edition, p. 985).

The condition is characterized by a lumpy or dimpled skin texture. The tissue often lacks firmness and elasticity. It is more prevalent in women than in men. It is not associated with obesity. The dimpling is believed to be caused by the swelling of fat cells which distend the skin along with the contraction of the vertical strands of fibrous tissue, both being attributed to decreased circulation and/or hormonal changes.

As further explanation, the protein fibers that make up the skin's connective tissue cross-link, causing the collagenous layer of tissue to lose elasticity and to harden, thereby inhibiting it's ability to absorb water and other essential body fluids. Due to decreased circulation, the tissue just below the skin accumulates waste materials that form gelatinous substances that harden into immovable structures. Another common term for this condition is “fibrosis”. The fatty tissue is pushed closer to the skin's surface in an irregular pattern that causes the dimpling appearance. A good example of this appearance is the skin of an orange.

The presence of “cellulite” is very distressing to some people. To cash in on this distress, alleged “anticellulite” products are sold through retail outlets, by mail, through multilevel companies, and through the Internet. Mostly useless, these treatments and products include “loofah” sponges; cactus fibers; special washcloths; horsehair mitts; creams and gels to “dissolve” cellulite; supplements containing vitamins; minerals and/or herbs; bath liquids; massagers; rubberized pants; exercise books; brushes; rollers; body wraps; and toning lotions. Many salons offer treatment with electrical muscle stimulation, vibrating machines, inflatable hip-high pressurized boots, “hormone” or “enzyme” injections, heating pads, and massage. Some operators claim that 5 to 15 inches can be lost in one hour. A series of treatments can cost hundreds of dollars.

In short, millions upon millions of dollars are spent each year (principally by women) on “treatments” for cellulite, little of which have measurable or perceptible, long term results.

In view of the above, it would be highly desirable and (to some) literally psychologically therapeutic to provide a truly effective treatment of lipoedema or “cellulite.”

### SUMMARY OF THE INVENTION

It is an object of the present invention to provide an improved treatment regimen for cellulite.

It is another object of the present invention to provide an improved treatment regimen for cellulite, which treatment regimen obviates any need or indication for surgery, steroid treatment, or other treatment modalities with known potential side effects and/or treatment risks or discomfort.

It is another object of the present invention to provide an improved treatment regimen for cellulite, which treatment regimen is based on the use of a pharmacological agent known to have minimal potential side effects.

It is another object of the present invention to provide an improved treatment regimen for cellulite, which treatment regimen obviates, at least in part, the need for all but a mere topical application of a therapeutic medicament.

In satisfaction fees and related objects, the present invention provides for the treatment of cellulite through the periodic topical application of a calcium channel blocker agent which is combined with an agent for facilitating transdermal penetration thereof.

It is proposed that when the referenced medicament is applied topically to the affected areas, the absorption enhancers incorporated with the active ingredient (a calcium channel blocker agent) causes the active ingredient to be absorbed into the fibrous tissue. The calcium channel blocker prevents circulating divalent calcium from entering the affected cells and causes a maturation of existing fibrobasts of collagenase. This resulting active collagenase then causes a remodeling of the fibrotic tissue into healthy tissue with improved circulation, improved elasticity, and a resulting smooth appearance.

### DETAILED DESCRIPTION OF THE PREFERRED EMBODIMENT

In the preferred embodiment of the present medicament, and in the medicament upon which the associated method are based, the primary active ingredient is Verapamil Hydrochloride, USP (a diphenylalkylamine). However, it should be understood that other calcium channel blocker agents (topically applied in a similar composition to that taught herein) provide similar, if not indistinguishable results when treating a variety of amber and fibrotic tissue manifestations. Therefore, it is contemplated that the transdermal administration through topical application of calcium channel blocker agents suspended in a penetration enhancing agent will, regardless of the species of the calcium channel blocker agent(s), exhibit efficacy in treating cellulite. Today, patient trials have been conducted through the use of the herein described verapamil-based topical gels, with very favorable and prompt results (visible changes observed within a matter of weeks). Other calcium channel blocker agents which could supplement or substitute for verapamil include benzothiazepines (Diltiazem, for example), dihydropyridines (Amlodipine, Felodipine, Isradipine, Nicardipine, Nifedipine, Nimodipine, or Nisoldipine), and the fast sodium inward channel inhibitor—Bepridil.

I. Preparation.

The preferred Verapamil-based gels of the present invention (in exemplary 10% and 15% percent strengths) may be prepared according to the following disclosure and protocol, with variations appropriate to a desired scale of production as will be apparent to persons skilled in the production of pharmaceutical preparations:

A. Constituents of Preferred Embodiment of Topical Verapamil Gel 10% and 15%

| Ingredients | 10% (% W/W) | 15% (% W/W) |
|---|---|---|
| Verapamil | 10.0 | 15.0 |
| Ethoxydiglycol | 14.0 | 19.5 |
| Propylene Glycol | 0.5 | 0.5 |
| Butylated Hydroxy Toluene (BHT) | 0.1 | 0.1 |
| Lecithin Soya Granular | 13.1 | 13.1 |
| Isopropyl Myristate | 13.1 | 13.1 |
| Sorbic Acid | 0.09 | 0.09 |
| Pluronic F127 | 9.8 | 11.6 |
| Potassium Sorbate | 0.15 | 0.12 |
| Disodium Edetate | 0.01 | 0.01 |
| Purified Water | 39.15 | 26.88 |

B. Topical Verapamil 15% (To Make 3000 Gm).

| Ingredients | Quantity |
|---|---|
| Verapamil HCI USP | 450.00 Gm |
| Ethoxydiglycol Reagent | 585.0 Gm |
| Lecithin/Isopropyl Myristate Solution | 790.0 Gm |
| Butylated Hydroxytolune NF (BHT) | 3.0 Gm |
| Edetate Disodium USP | 0.30 Gm |
| Propylene Glycol USP | 15.0 Gm |
| Pluronic Gel 30% | 1,156.7 Gm |

Instructions: Dissolve verapamil in ethoxydiglycol and propylene glycol with the aid of heat (90-100 degrees C.). Stir during this dissolving step. When the solution is clear, weigh to ascertain the amount of evaporation. Add the amount lost to evaporation back as ethoxydiglycol. Immediately add the lecithin/isopropyl myristate and BHT and stir well. Weigh the PLO 30% into a plastic container, add edetate disodium and stir gently to dissolve edetate disodium. Avoid foaming with stirring. Gently add the verapamil phase to the PLO phase, avoiding the incorporation of air. Stir for 10 minutes using a 3 inch mixing blade at 3100 rpm. Dispense in 30 Gm glaminate ointment tubes.

C. Topical Verapamil 10% (To Make 3000 Gm).

| Ingredients | Quantity |
|---|---|
| Verapamil HCI USP | 300.00 Gm |
| Ethoxydiglycol Reagent | 420.0 Gm |
| Lecithin/Isopropyl Myristate Solution | 790.0 Gm |
| Butylated Hydroxytolune NF (BHT) | 3.0 Gm |
| Edetate Disodium USP | 0.30 Gm |
| Propylene Glycol USP | 15.0 Gm |
| Pluronic Gel 30% | 1,471.7 Gm |

Instructions: Dissolve verapamil in ethoxydiglycol and propylene glycol with the aid of heat (90-100 degrees C.). Stir during this dissolving step. When the solution is clear, weigh to ascertain the amount of evaporation. Add the amount lost to evaporation back as ethoxydiglycol. Immediately add the lecithin/isopropyl myristate and BHT and stir well. Weigh the PLO 30% into a plastic container, add edetate disodium and stir gently to dissolve edetate disodium. Avoid foaming with stirring. Gently add the verapamil phase to the PLO phase, avoiding the incorporation of air. Stir for 5 minutes using a 3 inch mixing blade at 3100 rpm. Dispense in 30 Gm glaminate ointment tubes.

D. Pluronic Gel 20% (To Make 3000 Gm)

| Ingredients | Quantity |
|---|---|
| Pluronic F127 NF (Poloxamer 407) | 600.00 Gm |
| Potassium Sorbate NF | 9.00 Gm |
| Water (Sterile for Irrigation) qs to | 3,000.00 Gm |

Directions: Prepare a pluronic gel by combining the potassium sorbate and pluronic F 127 and bringing to a total weight of 3,000 Gm. with cold (refrigerated) sterile water. Make sure that all the granules are wet, and place in a refrigerator. Mixture will form a clear solution over 24-48 hours.

Alternate Procedure: The above mixture can be uniformly mixed with a mixing blade. It will take on the appearance of beaten egg whites. When placed in the refrigerator it will form a clear solution much faster, usually overnight. The above solution will solidify into a clear gel at room temperature.

E. Pluronic Gel 30% (To Make 2000 Gm).

| Ingredients | Quantity |
|---|---|
| Pluronic F 127 NF (Poloxamer 407) | 600.00 Gm |
| Potassium Sorbate NF | 6.00 Gm |
| Water (Sterile for Irrigation) qs to | 2,000.00 Gm |

Instructions: Prepare a pluronic gel by combining the potassium sorbate and pluronic F 1 27 and bringing to a total weight of 2,000 Gm. with cold (refrigerated) sterile water. Make sure that all the granules are wet, and place in a refrigerator. Mixture will form a clear solution over 24-48 hours.

Alternate Procedure: The above mixture can be uniformly mixed with a mixing blade. It will take on the appearance of beaten egg whites. When placed in the refrigerator it will form a clear solution much faster, usually overnight. The above solution will solidify into a clear gel at room temperature.

F. Lecithin/Isopropyl Myristate Solution (To Make 3000 Gm).

| Ingredients | Quantity |
|---|---|
| Lecithin Soya Granular | 1,494.0 Gm |
| Isopropyl Myristate NF | 1,494.0 Gm |
| Sorbic Acid NF Powder | 9.90 Gm |

Instructions: Disperse lecithin and sorbic acid in isopropyl myristate. Allow to stand at room temperature until a liquid of syrup consistency forms. Stir well and store in a light protected container.

G. Alternative Formulations.

It is to be understood that the above formulations and preparation methodologies are merely those which are believe to be ideal based on current experience and knowledge. Cellulite may be successfully treated using formulations like, or substantially equivalent to those taught in the U.S. Pat. No. 6,031,005 the entirety of the disclosure of which patent is incorporated herein by reference as if set forth herein verbatim. The addition of constituents in the present medicaments over those taught in the reference to patent are included for, and relate principally to product stability, not to efficacy. Therefore, the combination of one or more calcium channel blocker agents and any penetration enhancing agent for facilitating transdermal delivery of active ingredients in the treatment of cellulite is within the scope of the present invention and of the appended claims.

II. Use of Preparations.

The choice of strengths of the topical calcium channel blocker gels taught above will depend on the experience of the clinician, as will the duration of treatment. The presently believe to, preferred mode involves the use of the above taught 10% concentration, but some clinicians may find that use of a 15% concentration (or other concentration) may provide more desirable results with certain patients.

The patient is to be instructed to massage the topical medication into the skin sufficiently to cover the entire affected area(s) and just beyond the margins thereof. The topical medication contains a 10% concentration of the active ingredient, the calcium channel blocker. The medicament may be applied twice daily for fastest results, but ideally should be applied at least once daily. If applied twice daily, the applications should be in the morning and at bedtime.

Just before applying the medication the patient is instructed to massage the affected area well in order to improve blood circulation to the affected areas. This can be accomplished with a mechanical vibrator or a tool as simple as a baker's rolling pen.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

I claim:

1. A medicament for use in the treatment of cellulite comprising:
a carrier host agent for facilitating transdermal application of a calcium channel blocker agent to an affected bodily structure; and
a calcium channel blocker agent suspended in said carrier host agent.

2. The medicament of claim 1 further comprising:
an antioxidant agent suspended in said carrier host agent for preventing the oxidation of active ingredients of said medicament.

3. The medicament of claim 1 wherein said calcium channel blocker agent is verapamil.

4. The medicament of claim 1 wherein said medicament comprises:
verapamil;
a lecithin/isopropyl myristate solution;
butylated hydroxy toluene;
pluronic F127;
an antioxidant agent suspended in said carrier host agent for preventing the oxidation of active ingredients of said medicament; and
water.

5. The medicament of claim 4 further comprsing:
Edetate disodium.

6. The medicament of claim 4 further comprsing:
Propylene glycol.

7. The medicament of claim 5 further comprsing:
Propylene glycol.

8. A method for treating a cellulite comprising the steps of:
selecting a medicament comprising:
carrier host agent for facilitating transdermal application of a calcium channel blocker agent to a bodily structure having an accumulation of cellulite;
a calcium channel blocker agent suspended in said carrier host agent; and
periodically, topically applying a therapeutic dosage of said medicament to said accumulation of cellulite for sufficient time to effect a desired level of reduction of palpable said cellulite.

9. The method of claim 8 wherein said calcium channel blocker agent is verapamil.

10. The method of claim 8 wherein said calcium channel blocker agent is selected from the chemical groups of consisting of diphenylalkylamine, benzothiazepines, or dihydropyridines.
